(19) United States
(12) Patent Application Publication
(10) Pub. No.: US 2022/0181031 A1
(43) Pub. Date: Jun. 9, 2022

(54) RISK STRATIFICATION BASED ON INFECTION RISK AND AIR POLLUTION EXPOSURE

(71) Applicant: KONINKLIJKE PHILIPS N.V., EINDHOVEN (NL)

(72) Inventors: Huibin WEI, Eindhoven (NL); Jennifer CAFFAREL, Eindhoven (NL); Jarno Mikael RIISTAMA, Waalre (NL); Declan Patrick KELLY, Shanghai (CN); Yifei YANG, Shanghai (CN)

(21) Appl. No.: 17/421,593

(22) PCT Filed: Jan. 9, 2020
(86) PCT No.: PCT/EP2020/050350

§ 371 Date: Jul. 8, 2021

(30) Foreign Application Priority Data
Jan. 9, 2019 (CN) PCT/CN2019/071014

(51) Int. Cl. G16H 50/30

(52) U.S. Cl. CPC G16H 50/30

(57) ABSTRACT

A method for calculating an augmented health risk score by a health risk system including a processor, including: receiving, by the processor, CO2 level data and location data for a user; receiving, by the processor, location data for the user; determining, by the processor, when the user is outdoors based upon the received CO2 level data and calculating an outdoor pollution exposure based upon pollution data for the user's location; determining, by the processor, when the user is indoors based upon the received CO2 level data, calculating a ventilation rate based upon the received CO2 level data and the local outdoor pollution levels, and calculating an indoor pollution exposure based upon pollution data for the user's location; and calculating, by the processor, the augmented health risk score based upon the outdoor pollution exposure, indoor pollution exposure, a medical history of the user, and demographic information of the user.

### TECHNICAL FIELD

Various exemplary embodiments disclosed herein relate generally to risk stratification based on infection risk and air pollution exposure.

### BACKGROUND

Air pollution levels remain dangerously high in many parts of the world. Data from the World Health Organization (WHO) shows that 9 out of 10 people breathe air containing high levels of pollutants. WHO estimates that, one in eight of total global deaths (7 million people every year) was due to air pollution exposure. Ambient air pollution alone caused approximately 4.2 million deaths in 2016; household air pollution from kerosene and solid cooking fuels caused an estimated 3.8 million deaths in the same period. WHO recognizes that air pollution is a critical risk factor for non-communicable diseases (NCDs), causing an estimated one-quarter (24%) of all adult deaths from heart disease, 25% from stroke, 43% from chronic obstructive pulmonary disease and 29% from lung cancer.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for calculating an augmented health risk score by a health risk system including a processor, including: receiving, by the processor, CO2 level data and location data for a user; receiving, by the processor, location data for the user; determining, by the processor, when the user is outdoors based upon the received CO2 level data and calculating an outdoor pollution exposure based upon pollution data for the user's location; determining, by the processor, when the user is indoors based upon the received CO2 level data, calculating a ventilation rate based upon the received CO2 level data and the local outdoor pollution levels, and calculating an indoor pollution exposure based upon pollution data for the user's location; and calculating, by the processor, the augmented health risk score based upon the outdoor pollution exposure, indoor pollution exposure, a medical history of the user, and demographic information of the user.

Various embodiments are described, wherein the ventilation rate is calculated as:

$${Q}_{O} = \frac{G}{{C}_{in , ss} - {C}_{out}} ,$$

where Q₀ is the indoor air ventilation rate per person, G is the CO2 generation rate per person, C_{in,ss} is received CO2 level, and Cₒᵤₜ is an outdoor CO2 level based upon the user's location.

Various embodiments are described, further including calculating an infection risk value based upon the calculated ventilation rate, wherein the augmented health risk score is further based upon calculated infection risk value.

Various embodiments are described, wherein infection risk value is calculated as:

$${r}_{I} = 1 - {e}^{- \frac{Iqpt}{{Q}_{0}}} ,$$

where r_{I} represents the possibility of getting infection by the user, Q₀ represents the calculated ventilation rate, I represents number of people in an indoor area with the user, t represents the time duration that the user is in the indoor area, p is the pulmonary ventilation rate of user, and q represents the infection rate at the population level.

Various embodiments are described, further including providing the user with an alert based upon their location when their augmented health risk score exceeds a threshold level.

Various embodiments are described, further including providing the user with a recommended intervention based upon their health conditions and augmented health risk score.

Various embodiments are described, further including receiving from a user interface indoor activity information from the user, wherein the augmented health risk score is further based upon the received indoor activity information.

Various embodiments are described, further including receiving from a device in the user's vicinity indoor pollution information, wherein the augmented health risk score is further based upon the received device pollution information.

Various embodiments are described, wherein the device is one of a particulate matter sensor, a vacuum cleaner, a stove, a deep fryer, and gas sensor.

Various embodiments are described, further including receiving CO2 level data and pollution data from other users.

Various embodiments are described, further including monitoring a user's activity over time to determine a user's routine, wherein the user's routine is used to calculate the users augmented health score.

Further various embodiments relate to a method for calculating augmented health risk scores for patients by a health management system including a processor, including: receiving, by the processor, CO2 level data and location data for a plurality of patients; receiving, by the processor, location data for the plurality of patients; determining, by the processor, when the patients are outdoors based upon the received CO2 level data and calculating an outdoor pollution exposure for each patient based upon pollution data for the patient's location; determining, by the processor, when the patients are indoors based upon the received CO2 level data, calculating a for each patient a ventilation rate based upon the received CO2 level data and the local outdoor pollution levels, and calculating an indoor pollution exposure for each based upon pollution data for the patient's location; and calculating, by the processor, the augmented health risk score for each patient based upon the outdoor pollution exposure, indoor pollution exposure, a medical history of the patient, and demographic information of the patient.

Various embodiments are described, further including providing a patient with an alert based upon their location when their augmented health risk score exceeds a threshold level.

Various embodiments are described, further including providing a patient with a recommended intervention based upon their health conditions and augmented health risk score.

Various embodiments are described, further including monitoring a patient's activity over time to determine a patient's routine, wherein the patient's routine is used to calculate the users augmented health score.

Further various embodiments relate to a non-transitory machine-readable storage medium encoded with instructions for calculating an augmented health risk score by a health risk system including a processor, including: instructions for receiving CO2 level data and location data for a user; instructions for receiving location data for the user; instructions for determining when the user is outdoors based upon the received CO2 level data and calculating an outdoor pollution exposure based upon pollution data for the user's location; instructions for determining when the user is indoors based upon the received CO2 level data, calculating a ventilation rate based upon the received CO2 level data and the local outdoor pollution levels, and calculating an indoor pollution exposure based upon pollution data for the user's location; and instructions for calculating the augmented health risk score based upon the outdoor pollution exposure, indoor pollution exposure, a medical history of the user, and demographic information of the user.

Various embodiments are described, wherein the ventilation rate is calculated as:

$${Q}_{O} = \frac{G}{{C}_{in , ss} - {C}_{out}} ,$$

where Q₀ is the indoor air ventilation rate per person, G is the CO2 generation rate per person, C_{in,ss} is received CO2 level, and Cₒᵤₜ is an outdoor CO2 level based upon the user's location.

Various embodiments are described, further including instructions for calculating an infection risk value based upon the calculated ventilation rate, wherein the augmented health risk score is further based upon calculated infection risk value.

Various embodiments are described, wherein infection risk value is calculated as:

$${r}_{I} = 1 - {e}^{- \frac{Iqpt}{{Q}_{0}}} ,$$

where r_{I} represents the possibility of getting infection by the user, Q₀ represents the calculated ventilation rate, I represents number of people in an indoor area with the user, t represents the time duration that the user is in the indoor area, p is the pulmonary ventilation rate of user, and q represents the infection rate at the population level.

Various embodiments are described, further including instructions for providing the user with an alert based upon their location when their augmented health risk score exceeds a threshold level.

Various embodiments are described, further including instructions for providing the user with a recommended intervention based upon their health conditions and augmented health risk score.

Various embodiments are described, further including instructions for receiving from a user interface indoor activity information from the user, wherein the augmented health risk score is further based upon the received indoor activity information.

Various embodiments are described, further including instructions for receiving from a device in the user's vicinity indoor pollution information, wherein the augmented health risk score is further based upon the received device pollution information.

Various embodiments are described, wherein the device is one of a particulate matter sensor, a vacuum cleaner, a stove, a deep fryer, and gas sensor.

Various embodiments are described, further including instructions for receiving CO2 level data and pollution data from other users.

Various embodiments are described, further including instructions for monitoring a user's activity over time to determine a user's routine, wherein the user's routine is used to calculate the users augmented health score.

Further various embodiments relate to a non-transitory machine-readable storage medium encoded with instructions for calculating augmented health risk scores for patients by a health management system including a processor, including: instructions for receiving CO2 level data and location data for a plurality of patients; instructions for receiving location data for the plurality of patients; instructions for determining when the patients are outdoors based upon the received CO2 level data and calculating an outdoor pollution exposure for each patient based upon pollution data for the patient's location; instructions for determining when the patients are indoors based upon the received CO2 level data, calculating a for each patient a ventilation rate based upon the received CO2 level data and the local outdoor pollution levels, and calculating an indoor pollution exposure for each based upon pollution data for the patient's location; and instructions for calculating the augmented health risk score for each patient based upon the outdoor pollution exposure, indoor pollution exposure, a medical history of the patient, and demographic information of the patient.

Various embodiments are described, further including instructions for providing a patient with an alert based upon their location when their augmented health risk score exceeds a threshold level.

Various embodiments are described, further including instructions for providing a patient with a recommended intervention based upon their health conditions and augmented health risk score.

Various embodiments are described, further including instructions for monitoring a patient's activity over time to determine a patient's routine, wherein the patient's routine is used to calculate the users augmented health score.

### BRIEF DESCRIPTION OF THE DRAWINGS

In order to better understand various exemplary embodiments, reference is made to the accompanying drawings, wherein:

FIG. 1 illustrates a flow of how to measure infection risk and pollution exposure, to then perform a health risk evaluation, and then to determine any needed intervention;

FIG. 2 illustrates a user interface that may be used to collect information regarding indoor activities contributing to indoor pollution; and

FIG. 3 illustrates a health management system that incorporates pollution information to produce an augmented health risk score.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

It has been estimated in various studies that, in the United States, a mean reduction in PM2.5 (the density of atmospheric particulate matter that have a diameter of less than 2.5 micrometers) of 3.9 μg/m³ would prevent 7978 heart failure hospitalizations and save a third of a billion US dollars a year. The American Heart Association (AHA) stated that, although the dangers to one individual at any single time point may be small, the public health burden derived from this ubiquitous risk is enormous. Short-term increases in PM2.5 levels lead to the early mortality of tens of thousands of individuals per year in the United States alone. So it is very useful for both of individual and population health managers to have the information of pollution exposure for the high-risk group of people.

Not only particular matter, but the other air attributes from outdoor pollution, such as O₃, NO₂, SO₂, could also cause negative effect on health. Many studies have shown the health effect of these outdoor pollutants, which mainly come from industry, energy supply, transportation, sunlight, etc. A meta-analysis revealed that every 10 ppb increase of SO₂ could cause 2.9% increase of lung infection risk. A 10 μg/m³ increase could cause 3.4% increased risk of hypertension.

It is very important to avoid pollution exposure, especially for sensitive groups of people, such as infants, the elderly, pregnant women, and patients with respiratory/cardiovascular disease, cancer, or other severe diseases. Furthermore, for sensitive groups of people, respiratory infection is another big issue which may cause more severe complications or increase the severity of a disease. The risk of getting infected increases with poor ventilation and more infection sources. Exposure to air pollution will further increase the chance of getting an infection.

However, because peoples' activity includes both indoor and outdoor activities and because outdoor pollution gets indoors through ventilation, there is no existing solution for tracking an individual's risk of getting an infection and the pollution exposure related to above mentioned pollutants.

It is very useful to track an individual's infection risk and pollution exposure which contributes to the increased risk of certain diseases, and a direct method would be wearing small sensors which can detect target infection source and pollutants. However, wearable sensors including bacteria/viruses, PM2.5, PM10, SO₂, NO₂, and O₃ are not now available or mature in the market for the following reasons. For an infection detection sensor, because of the various types of bacteria/viruses and the low aerosol concentration of these bacteria/viruses, it is not possible to monitor them real-time using a small wearable sensor. For a wearable particulate matter (PM) sensor, because a steady air flow is required to ensure accurate results, which is normally provided by small fan, size is an obstacle making a wearable PM sensor impractical. A laser detector may be used for particle number counting to implement a PM sensor, but such a PM sensor becomes expensive in order to achieve the desired detection accuracy and small size. For gas sensors (SO₂, NO₂, O₃), the sensitivity, selectivity, response time, and accuracy would need to be balanced with price for a wearable gas sensor solution.

An alternative method to track an individuals' exposure to outdoor pollution is to track a user's position using GPS, and such measured location may be used to determine the local pollution levels from various databases and sensors that are publicly available. However, when people stay indoor with door/window open, or stay in a space with a badly filtered mechanical ventilation system, they are still exposed to outdoor pollution, which is not accounted for using GPS location tracking.

Furthermore, in population health management, care managers are taking care of a large group of patients, e.g., based on one zip-code area. However, because individuals' daily activities differ from each other quite a lot, only using outdoor pollutant data in the region is not sufficient for risk stratification and management of the patients. Also, for the care managers, one of the main desires is getting the personalized pollution risk evaluation for each patient.

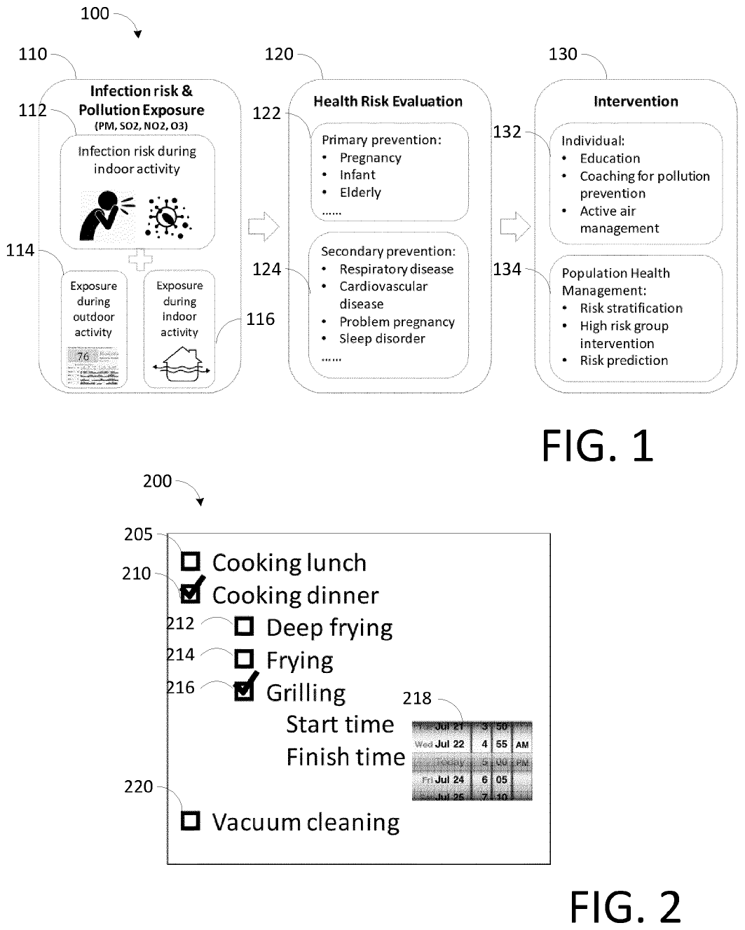

An embodiment of a solution to monitor an individual's infection risk and exposure to outdoor sourced pollutants (i.e., PM, SO₂, NO₂, O₃) via an indirect approach to avoid multiple sensor integration and high cost will now be described. This pollution exposure data could be further used for health risk evaluation and corresponding intervention at both the individual and population health level. FIG. 1 illustrates a flow of how to measure infection risk and pollution exposure, to then perform a health risk evaluation, and then to determine any needed intervention. The health risk evaluation system 100 has a risk and exposure module 110, a health risk evaluation module 120, and an intervention module 130. The risk and exposure module 110 determines the infection risk and pollution exposure for an individual or a group of individuals. This includes determining the infection risk during indoor activities 112. Also the exposure during outdoor activities 114 may be determined. Finally, the pollution exposure during indoor activities may be determined as well. The health risk evaluation module 120 takes the various information from the risk and exposure module 110 to evaluate the health risk for an individual or a group of individuals. This evaluation may focus on primary prevention 122 that would focus on those who are pregnant, infants, the elderly, or other high risk individuals or groups. The evaluation may also focus on secondary prevention 124 where those with specific diseases or disorders are considered, such as those with respiratory disease, cardiovascular disease, problem pregnancies, sleep disorders, etc.

Finally, the intervention model 130 takes the health risk evaluation and may then provide recommended interventions for individuals 132 or to help with population health management 132 e.g., by providing the care manager with an overview of the exposure in their population. Interventions for individuals 132 may include education, coaching strategies for pollution avoidance, active air management, etc. Population health management may include determining risk stratification, high risk group interventions, risk prediction, etc.

The health risk evaluation system to monitor infection risk and pollution exposure may include the following elements. A low cost, small, and fast responding gas sensor (i.e., a CO₂ sensor), which could be provided as a wearable sensor, sensor to be attached to clothing, or integrated on a body-worn health device, watch, or smart phone. From the detected CO₂ concentration, the health risk evaluation system may decide whether user is outdoors or indoors, and what the indoor ventilation rate is.

The health risk evaluation system may also include a GPS sensor or another sensor to determine the location of a user in order to localize where the measurement equipment is located. When the location may be determined otherwise, e.g., with a smartphone or other device associated with the user using the gas sensor, this might not be necessary.

The health risk evaluation system may also obtain information regarding major airborne infectious agents and the infection rate at the population level from e.g., Center of Disease Control (CDC) or a similar system. The health risk evaluation system may also obtain outdoor pollution data based on the nearest measuring station to the target individual.

The health risk evaluation system may also include a user interface for collecting certain user inputs, giving feedback or intervention coaching. Finally, the health risk evaluation system may also include other connected devices, if present, to further improve the accuracy of the evaluation (i.e., connected intelligent mask, smart watch, smart home appliances, air purifier, floor vacuum, etc.) More detail will now be provided for these various elements.

The detected CO₂ concentration measured by a wearable CO₂ sensor may be used to evaluate the user's surrounding environment: outdoor; indoor with high ventilation; or indoor with low ventilation. The following logic may be applied to the CO₂ data to identify the environment where the user is located.

When the detected CO₂ concentration is approximately equal to the local outdoor CO₂ concentration (a typical number is 400 ppm), the user is identified as being exposed to outdoor air or indoor air with the same constituent parts as outdoor air. The user's pollution exposure level is determined to be equal to the outdoor air pollution level.

When the detected CO₂ concentration is higher than outdoor CO₂ concentration, the user is identified as being indoors with a certain ventilation rate. The user's pollution exposure level from the outdoor sources could be calculated by outdoor pollution data and a measured ventilation rate.

Alternatively, the indoor air pollution exposure evaluation could be based upon a connected indoor air quality sensor/air purifier, a user's input of major activity causing indoor air pollution, etc. Such additional information may be combined with the measure of indoor ventilation and outdoor pollution levels to provide a more accurate determination of the air pollution present in an indoor area.

The relationship between CO₂ concentration and ventilation rate has been discussed in ASHRE Standard 62 [American Society for Heating, Refrigerating, and Air-Conditioning Engineers; 1981], in which the steady-state equation is presented as:

$${Q}_{O} = \frac{G}{{C}_{in , ss} - {C}_{out}} \qquad ( 1 )$$

where Q₀ is the indoor air ventilation rate per person, G is the CO₂ generation rate per person, C_{in,ss} is the steady-state indoor CO₂ concentration, and Cₒᵤₜ is the outdoor CO₂ concentration.

In the equation 1, the measured CO₂ concentration for the wearable CO₂ sensor may be used for C_{in,ss}, and Cₒᵤₜ is a known value take from the local outdoor CO₂ concentration measurements. G may be obtained by user's input identifying the other individuals in the indoor area and a machine learning algorithm. An applicable approach may that the gender, age, and weight of user's family members are pre-recorded in the health risk evaluation system, which will determine the CO₂ generation rate (parameter G in equation 1, assuming mainly light activities are taking place at home). When a CO₂ concentration higher than outdoor CO₂ concentration is detected, the health risk evaluation system may ask for user's input to indicate the participants at home and user may select family members who are currently at home. Alternatively, when various people in the indoor area are wearing the CO₂ sensor, the CO₂ sensors may automatically detect each other's proximity based on the location and potential other parameters such as connection to the same Wifi-base station. After a certain time period, a user's daily routine (e.g., 1 adult and 1 child during dinner time, 2 adults+a child during sleep time) may be determined and then recorded, and the user's input for the home participants will not be needed unless some unregular CO₂ pattern is detected.

The ventilation rate may be used to help determine an infection risk for a user. More specifically, the infection risk evaluation may use the calculated indoor ventilation rate, the number of people in the indoor area, and duration of the user's stay in the indoor area. The risk of getting an infection is much higher when one is exposed to a poorly ventilated room with many participants. The Wells-Riley model could be used to evaluate the risk of getting airborne infection:

$${r}_{I} = 1 - {e}^{- \frac{Iqpt}{{Q}_{0}}}$$

where r_{I} represents the possibility of getting infection for individual, Q₀ represents indoor air ventilation rate (m³/s), which may be determined as described above, I represents number of people in the indoor area, t represents the time duration that the individual is exposed in this environment, p is the pulmonary ventilation rate of individual (m³/s), and q represents the infection rate at the population level.

Once the infection rate is calculated, the user may receive feedback during/after they are exposed to a high infection risk environment. The feedback could be suggesting the following actions to the user: putting a mask, especially when user is in a crowded environment, i.e., subway in rush-hour, and if the mask is a connected intelligent mask, a certain risk reduction may be recorded in the health risk evaluation system; performing a nasal wash after returning back home; or any other intervention that would benefit the user based upon their specific exposure.

The health risk evaluation system may further make estimates of the pollution exposure of a user based upon various items such as PM including both outdoor sources (industry, transportation, etc.) and indoor sources (cooking, vacuum cleaning, etc.), while SO₂, NO₂, and O₃ are mainly from outdoor sources. If a connected PM sensor or a connected air purifier is available at home, this measured PM concentration data may be used in the evaluation to add to the pollution exposure from outdoor sources. If not available, a rough estimation of indoor source pollution may be estimated through user's input of indoor activity. For instance, at the end of the day, user is asked for the indoor activity of the day. FIG. 2 illustrates a user interface that may be used to collect information regarding indoor activities contributing to indoor pollution. The user interface 200 may include a list of activities that contribute to indoor pollution such as cooking lunch 205, cooking dinner 210, and vacuum cleaning 220. Cooking dinner if selected may include additional sub-activities such as deep frying 212, frying 214, and grilling 216. A further user interface element 218 may appear to allow the user to indicate a start and finish time for a selected activity. A rough PM concentration could be calculated by combining estimates based upon the selected activities, the ventilation rate, and the duration of the activities. Alternatively, if the user is wearing a smart watch for example, the watch might be able to distinguish certain activities automatically such as cooking and by the time of the day, the type of cooking could be also determined and utilizing the location information. The typical type of meal could also be estimated resulting in a typical PM emission level. For example, in the Netherlands, the typical lunch consists of bread, which emits much less PM than if something is being cooked on the stove, which might be a typical lunch elsewhere. Various known method may be used to combine these various bits of information to determine the indoor PM levels as well as pollution levels.

For a care manager who is responsible for population health management in a certain region, it is not easy to understand patients' daily activity in order to recognize the patients with higher-risk and the patients who need intervention. One approach would be applying the methods and health risk evaluation system described above for a certain period, e.g., 6 weeks, to understand their daily routine, activity pattern and pollution exposure of users, in order to achieve a better overview of the patient group and more accurate risk stratification with environmental and human behavior within consideration. By combining the information from sensors such as the wearable CO₂ sensors and location sensors, external and contextual information, clinical risk scores (e.g., cardiovascular risk, pulmonary risk, asthma risk, etc.) may be updated for the individuals being monitored to take into account their personal exposition to indoor/outdoor pollutants.

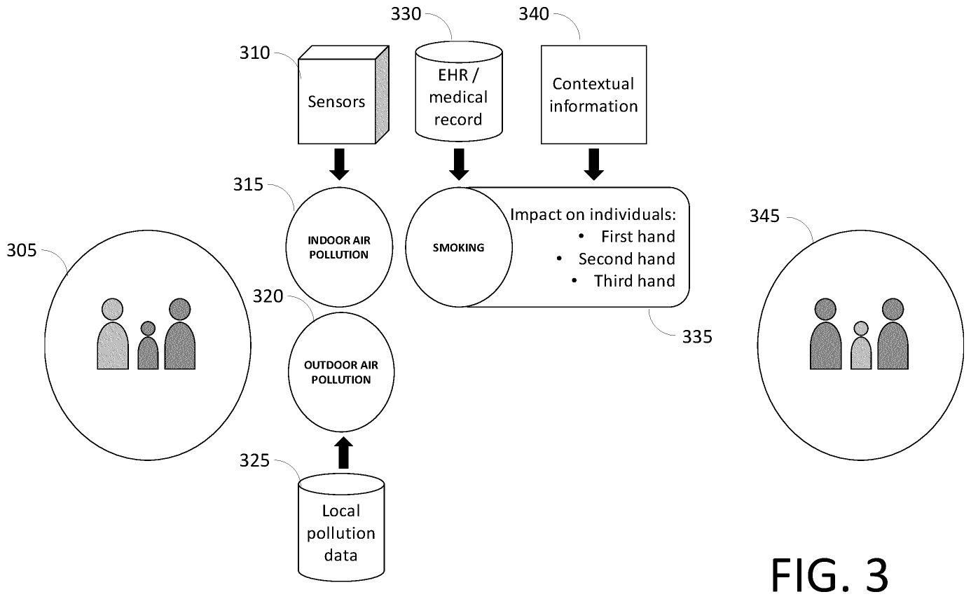

FIG. 3 illustrates a health management system that incorporates pollution information to produce an augmented health risk score. The health management system starts with an original health risk score 305 that may be based upon any health risks that the users, in this example a family, may have. Sensor 310 such as wearable CO₂ sensors, location sensors, or other sensors may collect information that may be used to determine indoor pollution exposure 314. Further, local pollution data 325 may be used along with location data to determine how local pollution levels affect the user. This will determine the outdoor pollution exposure 320 based upon location data indicating the amount of time spent outdoors. Also the indoor pollution exposure may be based in part on the outdoor pollution levels and the ventilation rate. Further, electronic health records (EHR) and medical records may be used to identify further pollution risks for the users, such as being a smoker or being around a smoker. Contextual information 340 will identify family members of smokes who will be affected by both second hand smoke and third hand smoke. These additional factors may be combined with the indoor pollution calculation and outdoor pollution calculations to update the original risk score to an augmented risk score. This augmented risk score may then be used by a care manager to consider various interventions that might be suggested to the user to improve their health. The original risk score is based only upon medical conditions and demographic information. This original risk score may be improved by augmenting it based upon the pollution information as described above. Also, the health risk score may include infection risk as well.

The various information collected by the health risk evaluation system may be used to crowd source information for population health management. The detected infection risk, ventilation rate, and pollution exposure level for a large group of users may be provided to a health care manager via the anonymous collection data from the users with wearable sensors or other sensors located in the region of interest. This collected information could provide information for the benefit of other users to provide more accurate health risk scores due to pollution and infection risks, which may help a user to decide, for example, if it is safe for the user to enter a certain space according to their own health status. This may also help users who do not have a wearable sensor, to roughly evaluate their infection risk and pollution exposure by using data collected from other users are in the same space or area.

In addition to the various sensors mentioned above, the health risk evaluation system may include a user application the runs on a user's computer, smart phone, tablet, etc. This application may be stand alone or web based. The user may enter the information regarding activities as described above. Also, the application may collect data from various sensors available around the user as well as collecting publicly available data such as pollution levels. This application would also have access to the user's other health and demographic information, and hence would be able to calculate the users augmented health risk score. This risk score may be presented to the user, but may also be used to create alerts for the user to indicate that they have entered a high risk area or have exceeded some risk threshold. The application could provide various interventions or actions to the user to avoid the risky situation or counter the increase risk. Also the user application may communicate with a central system that interfaces with a number of users to provide additional crowd sourced data to the user. This central system may also be the source of the publicly available pollution information for the user application. This application may even be used by users without their own wearable CO₂ sensor, by using crowdsourced data to determine their own risk based upon their location.

The health risk evaluation system may also include a provider application for health care providers. This application may be hosted on central servers or as a standalone application. The provider application may gather information for patients under the providers care. The various pollution risk and infection risk information may be collected and entered into the patients EHR to provide additional information to health care providers to consider when treating the patient. For example, the health care provider may determine that increased asthma problems may correlate with an increased exposure to pollution of the patient(s). The health care provider can then recommend certain interventions and behavior modification to prevent further asthma episodes. Further, the provider application may identify patients that are ask risk, bring those identified patients to the attention of the care provider, and the care provider may then reach out to the patient to help reduce their risk. Such a provider application could also help care providers track the progression of an infection throughout their patient population, that may be useful to understand the infection outbreak as well as allowing a care provider to provide warnings regarding increased infection rate.

The embodiments of a health risk evaluation system described herein solve the technological problem of accurately determining a user's health risk based upon pollution and infection risk. Various methods for determining health risk based upon medical conditions and demographic information are known, but these do not take into account pollution and infections risks to the user in their local environment. The health risk evaluation system uses a simple wearable gas detection sensor, such as a CO₂ sensor as well as location information, to further augment a user's health risk score based upon these factors. Further, local pollution information may be used to determine the user's pollution exposure along with any other sensor data that may be available. This allows for better treatment of patients, better prescriptions of interventions, and to provide risk warnings to the users and their care givers. The health risk evaluation system provides low cost personal pollution exposure evaluation system for users. The health risk evaluation system also provides health care managers a tool for risk stratification and prediction for patients that includes pollution exposure and infection risks.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for calculating an augmented health risk score by a health risk system including a processor, comprising:
receiving, by the processor, CO₂ level data and location data for a user;
receiving, by the processor, location data for the user;
determining, by the processor, when the user is outdoors based upon the received CO₂ level data and calculating an outdoor pollution exposure based upon pollution data for the user's location;
determining, by the processor, when the user is indoors based upon the received CO₂ level data, calculating a ventilation rate based upon the received CO₂ level data and the local outdoor pollution levels, and calculating an indoor pollution exposure based upon pollution data for the user's location; and
calculating, by the processor, the augmented health risk score based upon the outdoor pollution exposure, indoor pollution exposure, a medical history of the user, and demographic information of the user.

2. The method of claim 1, wherein the ventilation rate is calculated as:
$${Q}_{O} = \frac{G}{{C}_{in , ss} - {C}_{out}} ,$$
where Q₀ is the indoor air ventilation rate per person, G is the CO₂ generation rate per person, C_{in,ss} is received CO₂ level, and Cₒᵤₜ is an outdoor CO₂ level based upon the user's location.

3. The method of claim 1, further comprising calculating an infection risk value based upon the calculated ventilation rate, wherein the augmented health risk score is further based upon calculated infection risk value.

4. The method of claim 3, wherein infection risk value is calculated as:
$${r}_{I} = 1 - {e}^{- \frac{Iqpt}{{Q}_{0}}} ,$$
where r_{I} represents the possibility of getting infection by the user, Q₀ represents the calculated ventilation rate, I represents number of people in an indoor area with the user, t represents the time duration that the user is in the indoor area, p is the pulmonary ventilation rate of user, and q represents the infection rate at the population level.

5. The method of claim 1, further comprising providing the user with an alert based upon their location when their augmented health risk score exceeds a threshold level.

6. The method of claim 1, further comprising providing the user with a recommended intervention based upon their health conditions and augmented health risk score.

7. The method of claim 1, further comprising receiving from a user interface indoor activity information from the user, wherein the augmented health risk score is further based upon the received indoor activity information.

8. The method of claim 1, further comprising receiving from a device in the user's vicinity indoor pollution information, wherein the augmented health risk score is further based upon the received device pollution information.

9. The method of claim 8, wherein the device is one of a particulate matter sensor, a vacuum cleaner, a stove, a deep fryer, and gas sensor.

10. The method of claim 1, further comprising receiving CO₂ level data and pollution data from other users.

11. The method of claim 1, further comprising monitoring a user's activity over time to determine a user's routine, wherein the user's routine is used to calculate the users augmented health score.

12. A method for calculating augmented health risk scores for patients by a health management system including a processor, comprising:
receiving, by the processor, CO₂ level data and location data for a plurality of patients;
receiving, by the processor, location data for the plurality of patients;
determining, by the processor, when the patients are outdoors based upon the received CO₂ level data and calculating an outdoor pollution exposure for each patient based upon pollution data for the patient's location;
determining, by the processor, when the patients are indoors based upon the received CO₂ level data, calculating a for each patient a ventilation rate based upon the received CO₂ level data and the local outdoor pollution levels, and calculating an indoor pollution exposure for each based upon pollution data for the patient's location; and
calculating, by the processor, the augmented health risk score for each patient based upon the outdoor pollution exposure, indoor pollution exposure, a medical history of the patient, and demographic information of the patient.

13. The method of claim 12, further comprising providing a patient with an alert based upon their location when their augmented health risk score exceeds a threshold level.

14. The method of claim 12, further comprising providing a patient with a recommended intervention based upon their health conditions and augmented health risk score.

15. The method of claim 12, further comprising monitoring a patient's activity over time to determine a patient's routine, wherein the patient's routine is used to calculate the users augmented health score.

16. A non-transitory machine-readable storage medium encoded with instructions for calculating an augmented health risk score by a health risk system including a processor, comprising:
instructions for receiving CO₂ level data and location data for a user;
instructions for receiving location data for the user;
instructions for determining when the user is outdoors based upon the received CO₂ level data and calculating an outdoor pollution exposure based upon pollution data for the user's location;
instructions for determining when the user is indoors based upon the received CO₂ level data, calculating a ventilation rate based upon the received CO₂ level data and the local outdoor pollution levels, and calculating an indoor pollution exposure based upon pollution data for the user's location; and
instructions for calculating the augmented health risk score based upon the outdoor pollution exposure, indoor pollution exposure, a medical history of the user, and demographic information of the user.

17. The non-transitory machine-readable storage medium of claim 16, wherein the ventilation rate is calculated as:
$${Q}_{O} = \frac{G}{{C}_{in , ss} - {C}_{out}} ,$$
where Q₀ is the indoor air ventilation rate per person, G is the CO₂ generation rate per person, C_{in,ss} is received CO₂ level, and Cₒᵤₜ is an outdoor CO₂ level based upon the user's location.

18. The non-transitory machine-readable storage medium of claim 16, further comprising instructions for calculating an infection risk value based upon the calculated ventilation rate, wherein the augmented health risk score is further based upon calculated infection risk value.

19. The non-transitory machine-readable storage medium of claim 18, wherein infection risk value is calculated as:
$${r}_{I} = 1 - {e}^{- \frac{Iqpt}{{Q}_{0}}} ,$$
where r_{I} represents the possibility of getting infection by the user, Q₀ represents the calculated ventilation rate, I represents number of people in an indoor area with the user, t represents the time duration that the user is in the indoor area, p is the pulmonary ventilation rate of user, and q represents the infection rate at the population level.

20. The non-transitory machine-readable storage medium of claim 16, further comprising instructions for providing the user with an alert based upon their location when their augmented health risk score exceeds a threshold level.

21. The non-transitory machine-readable storage medium of claim 16, further comprising instructions for providing the user with a recommended intervention based upon their health conditions and augmented health risk score.

22. The non-transitory machine-readable storage medium of claim 16, further comprising instructions for receiving from a user interface indoor activity information from the user, wherein the augmented health risk score is further based upon the received indoor activity information.

23. The non-transitory machine-readable storage medium of claim 16, further comprising instructions for receiving from a device in the user's vicinity indoor pollution information, wherein the augmented health risk score is further based upon the received device pollution information.

24. The non-transitory machine-readable storage medium of claim 23, wherein the device is one of a particulate matter sensor, a vacuum cleaner, a stove, a deep fryer, and gas sensor.

25. The non-transitory machine-readable storage medium of claim 16, further comprising instructions for receiving CO₂ level data and pollution data from other users.

26. The non-transitory machine-readable storage medium of claim 16, further comprising instructions for monitoring a user's activity over time to determine a user's routine, wherein the user's routine is used to calculate the users augmented health score.

27. A non-transitory machine-readable storage medium encoded with instructions for calculating augmented health risk scores for patients by a health management system including a processor, comprising:
instructions for receiving CO₂ level data and location data for a plurality of patients;
instructions for receiving location data for the plurality of patients;
instructions for determining when the patients are outdoors based upon the received CO₂ level data and calculating an outdoor pollution exposure for each patient based upon pollution data for the patient's location;
instructions for determining when the patients are indoors based upon the received CO₂ level data, calculating a for each patient a ventilation rate based upon the received CO₂ level data and the local outdoor pollution levels, and calculating an indoor pollution exposure for each based upon pollution data for the patient's location; and
instructions for calculating the augmented health risk score for each patient based upon the outdoor pollution exposure, indoor pollution exposure, a medical history of the patient, and demographic information of the patient.

28. The non-transitory machine-readable storage medium of claim 27, further comprising instructions for providing a patient with an alert based upon their location when their augmented health risk score exceeds a threshold level.

29. The non-transitory machine-readable storage medium of claim 27, further comprising instructions for providing a patient with a recommended intervention based upon their health conditions and augmented health risk score.

30. The non-transitory machine-readable storage medium of claim 27, further comprising instructions for monitoring a patient's activity over time to determine a patient's routine, wherein the patient's routine is used to calculate the users augmented health score.
